(19) United States
(12) Patent Application Publication
(10) Pub. No.: US 2022/0028491 A1
(43) Pub. Date: Jan. 27, 2022

(54) BIOLOGICALLY INFORMED AND ACCURATE SEQUENCE ALIGNMENT

(71) Applicant: THE GENERAL HOSPITAL CORPORATION, Boston, MA (US)

(72) Inventors: Luca Pinello, Jamaica Plain, MA (US); Kendell Clement, Cambridge, MA (US)

(21) Appl. No.: 17/312,079

(22) PCT Filed: Dec. 13, 2019
(86) PCT No.: PCT/US2019/066167

§ 371 Date: Jun. 9, 2021

Related U.S. Application Data
(60) Provisional application No. 62/779,147, filed on Dec. 13, 2018.

(51) Int. Cl. G16B 30/10; C12N 15/10; C12N 9/22

(52) U.S. Cl. CPC G16B 30/10; C12N 9/22; C12N 15/102

(57) ABSTRACT

Systems and methods are provided for aligning a first sequence and a second sequence. A gap vector is populated with a plurality of gap penalty values representing a respective plurality of locations along the first sequence, such that a first gap penalty value associated with a first location of the plurality of locations is different than a second gap penalty value associated with a second location of the plurality of locations. For each of a set of possible alignments of the first sequence and the second sequence, a score is generated representing the fitness of each possible alignment. The score for each possible alignment is determined according to at least a match incentive, a mismatch penalty, and the gap vector. Z possible alignment of the set of possible alignments having a best score is selected as an alignment between the first sequence and the second sequence.

### RELATED APPLICATIONS

This application claims priority from U.S. Provisional Patent Application No. 62/779,147, filed Dec. 13, 2018, which is hereby incorporated by reference in its entirety.

### TECHNICAL FIELD

This invention relates to the field of expert systems, and more specifically, to systems and methods for aligning sequences.

### BACKGROUND

At the core of the study of genomic sequence similarity is the ability to compare between two sequences of characters which represent DNA nucleotides or protein residues. In the process of alignment, two sequences are ordered, and an alignment score can be calculated based on a scoring scheme which usually rewards alignments with many matches between the two sequences. Gaps can also be added between characters in either sequence to increase the number of matching characters. In a biological context, these gaps represent insertions or deletions, where DNA nucleotides or amino acid residues were deleted or inserted in one of the sequences. A number of algorithms have been developed for this purpose, including the Needleman Wunsch algorithm, the Gotoh algorithm, and the Smith Waterman algorithm.

### SUMMARY OF THE INVENTION

In accordance with an aspect of the present invention, a computer-implemented method is provided for aligning a first sequence and a second sequence. A gap vector is populated with a plurality of gap penalty values representing a respective plurality of locations along the first sequence, such that a first gap penalty value associated with a first location of the plurality of locations is different than a second gap penalty value associated with a second location of the plurality of locations. For each of a set of possible alignments of the first sequence and the second sequence, a score is generated representing the fitness of each possible alignment. The score for each possible alignment is determined according to at least a match incentive, a mismatch penalty, and the gap vector. The possible alignment of the set of possible alignments having a best score is selected as an alignment between the first sequence and the second sequence.

In accordance with another aspect of the present invention, a system includes a processor and a non-transitory computer readable medium storing instructions, executable by the processor, for aligning a first sequence and a second sequence. The instructions include a gap incentive component that populates a gap vector having a plurality of gap penalty values representing a respective plurality of locations along the first sequence according to expected locations for an action of a biological process applied to a subject associated with the first sequence and the second sequence. A sequence alignment component generates, for each of a set of possible alignments of the first sequence and the second sequence, a score representing the fitness of each possible alignment according to at least a match incentive, a mismatch penalty, and the gap vector and selects a possible alignment of the set of possible alignments having a best score as an alignment between the first sequence and the second sequence.

In accordance with yet another aspect of the present invention, a method is provided for determining an edit rate for a genome editing process. The genome editing process is performed on the subject. A plurality of read sequences are acquired from the subject. A gap vector is populated with a plurality of gap penalty values representing a respective plurality of locations along a reference sequence associated with the subject, such that a first gap penalty value associated with a first location of the plurality of locations is different than a second gap penalty value associated with a second location of the plurality of locations. Each of the plurality of read sequences is then aligned with a reference sequence.

Aligning each read sequence with the reference sequence includes generating, for each of a set of possible alignments of the reference sequence and the read sequence, a score representing the fitness of each possible alignment. The score for each possible alignment is determined according to at least a match incentive, a mismatch penalty, and the gap vector. A possible alignment of the set of possible alignments having a best score is selected as an alignment between the reference sequence and the read sequence. From the alignment of each read sequence with the reference sequence, it is determined if the read sequence represents a successful application of the genome editing process. An edit rate is determined across the plurality of read sequences indicative of the number of read sequences of the plurality of read sequences that represent successful applications of the genome editing process. The locations and types of genome editing modifications are then determined in the read sequences.

### BRIEF DESCRIPTION OF THE DRAWINGS

FIG. 1 illustrates one example of a method for determining an optimal alignment between two sequences;

FIG. 2 illustrates an example sequence alignment that benefits from the biological informed alignment of FIG. 1;

FIG. 3 illustrates one implementation of a method for aligning two sequences in accordance with an aspect of the present invention;

FIG. 4 illustrates a method for evaluating the effectiveness of a genome editing process in accordance with an aspect of the present invention;

FIG. 5 illustrates a system utilizing a biologically-informed sequence alignment to evaluate the results of a genome editing process in accordance with an aspect of the present invention; and

FIG. 6 illustrates a schematic block diagram illustrating an exemplary system of hardware components capable of implementing examples of systems and methods disclosed herein.

### DETAILED DESCRIPTION

The field of genome editing is revolutionizing the study and treatment of disease. Computational approaches have been created to design the targeting machinery for genome editing. The systems and methods described herein apply a biologically-informed method for sequence alignment to address for example the complementary problem of quantifying genome editing events for which the resulting DNA/RNA mutations have specific and non-random patterns. Sequence alignment refers to the matching of characters in sequences of DNA in a way that maximizes an alignment score, and a “sequence”, as used herein includes any genomic sequence, protein sequence or any biological sequence that can be measured for the purpose of alignment. Current aligners optimize a function called an alignment score that tries to find the best alignment based on a set of predefined rules. Finding the optimal alignment is a hard problem, and several algorithms have been designed to computationally find the best alignment. Based on the proposed rules different alignments can be reported for the same sequences. In some cases multiple solutions exists with exactly the same alignment score and disambiguation is necessary.

Described herein is a new alignment algorithm that specifically optimize the alignment process to prioritize and report biologically-likely alignments instead of reporting alignments bases on a generic set of rules that may be sub-optimal in a particular biological context. We illustrate an example of our approach in the genome editing context. Specifically, the algorithm identifies and quantifies genome editing events by aligning reads to a reference sequence taking into account biological information about the likelihood of a mutation occurring at each location in the sequence, and allowing this likelihood to be arbitrarily specified by a user. This alignment algorithm is employed in a software application to identify changes in DNA caused by genome editing tools and to quantify those editing events. A variety of scoring metrics have been developed for aligning sequences, but the scores are ambivalent to the location of insertion of gaps relative to the ends of the sequences. This reflects the biological assumption that insertions and deletions can occur throughout the sequences. Recently, with the advent of genome editing, genomic mutations can be targeted to a specific location in the genome. This dramatically changes the paradigm of sequence alignment which assumes that mutations occur equally likely throughout the sequence.

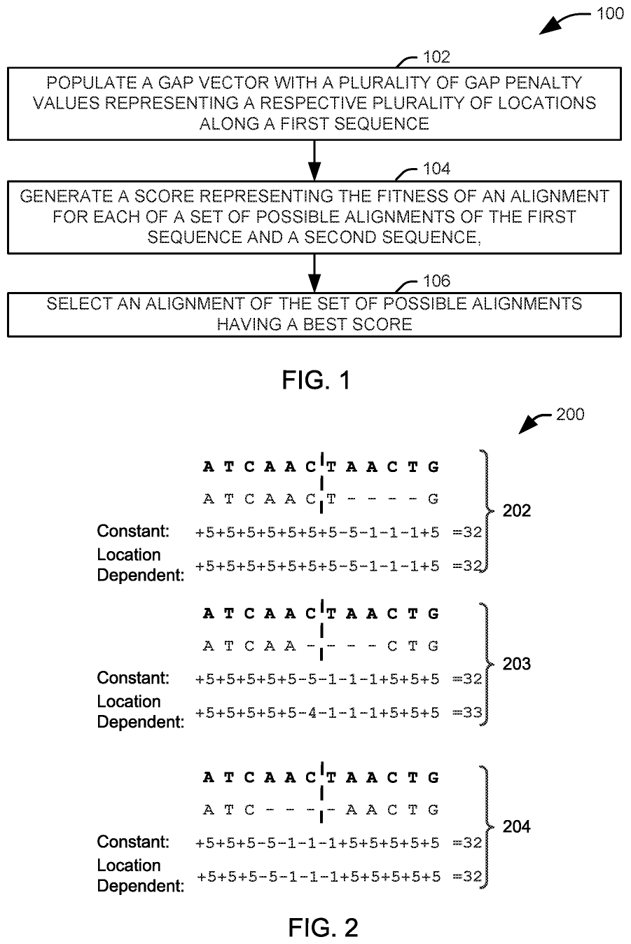

FIG. 1 illustrates one example of a method 100 for determining an optimal alignment between two sequences, referred to herein as A and B. The sequences can be acquired by any appropriate means including, but not limited to, illumina dye sequencing, ion semiconductor sequencing, Sequencing by Oligonucleotide Ligation and Detection (SOLID), and pyrosequencing. It will be appreciated that the steps of the method 100 are not limited to the sequence presented and are performed in parallel with millions of them.

At 102, a gap vector is populated with a plurality of gap penalty values representing a respective plurality of locations along the first sequence. It will be appreciated that sequencing algorithms generally utilize various incentives, such as a match incentive that rewards a pairing of corresponding elements of the two sequences when in a given alignment, a mismatch penalty that penalizes a pairing of corresponding elements of the two sequences when in a given alignment, and a gap penalty that penalizes a missing element in one sequence, referred to herein as a gap, that is paired with an element of the other sequence when in a given alignment. A gap can arise from the deletion of elements present in a reference sequence from a read sequence or from the addition of elements to the read sequence, such that gaps can appear in either sequence. Each of these incentives are selected as constant parameters for a given application.

In the illustrated method, however, the gap penalty varies according to the position of the gap in a given sequence, such that a first gap penalty value associated with a first location of the plurality of locations may be different than a second gap penalty value associated with a second location of the plurality of locations. In one implementation, the various elements of the gap penalty vector can be selected to represent biological information about the likelihood of a mutation occurring at each location in the sequence. For example, if a genome editing tool that creates a double-stranded break is targeted such that the cleavage enzyme is active immediately after the ‘CAAC’ sequence, gaps after a ‘CAAC’ sequence can be incentivized through a reduced gap penalty in the gap penalty vector. Through selection of the values of the gap penalty vector to match any applied genome editing process, the fitness of more biologically probable solutions in the algorithm can be enhanced. The specific values in the gap vector can be selected manually or determined via an automated process based on the known biological mechanism of an applied genome editing procedure.

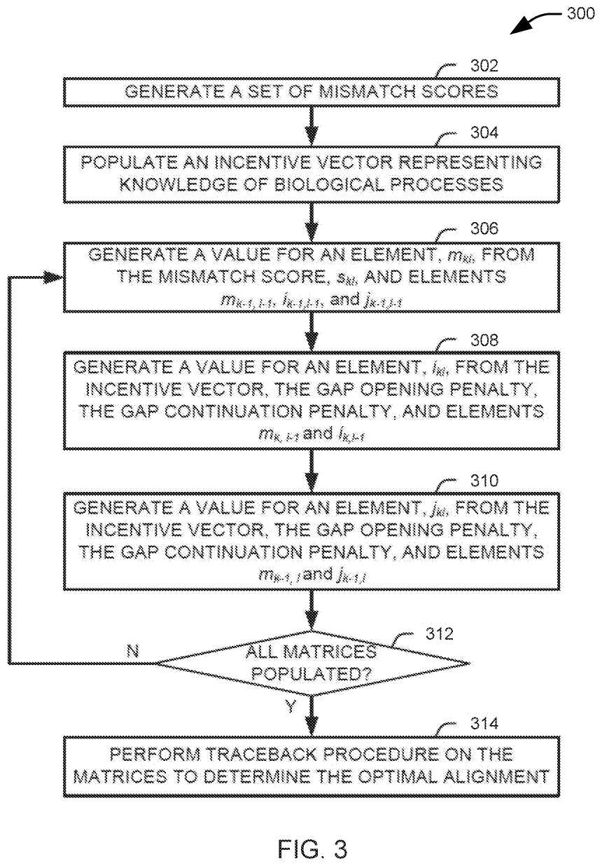

At 104, for each of a set of possible alignments of the first sequence and the second sequence, a score is generated representing the fitness of each possible alignment. The score for each possible alignment being determined according to at least a match incentive, a mismatch penalty, and the gap vector populated at 102. In one example, a matrix is iteratively populated such that each element of the matrix is a score representing the fitness of a possible alignment of at least a portion of the first sequence to a portion of the second sequence. One example of an algorithm for producing the scores for the possible alignment is provided in FIG. 3 below. It will be appreciated that a “score” as used herein, is arbitrary. In the descriptions herein, a larger score represents a better fitness for a given alignment, with mismatches and gaps being penalized with smaller or negative values. One of skill in the art will appreciate, however, that the sign of the values used for a given scoring system could be reversed or the values could be otherwise reassigned such that a match is represented by lower values. In such a case, wherever selection of a maximum value is described below, a minimum value would instead be selected.

At 106, a possible alignment of the set of possible alignments having a best score is selected as an alignment between the first sequence and the second sequence. The selected alignment can be displayed to a user and/or evaluated via another automated process to determine if it represents a successful instance of a biological process, such as a genome editing process, employed on a subject associated with the first sequence and the second sequence, or to characterize the modifications in some other way. In the example utilizing an iteratively populated matrix in 104, the possible alignment can be performed via a traceback procedure of the matrix that determines an optimal alignment of the first sequence to the second sequence given the contents of the matrix. The selected alignment can then be displayed to a user at an associated display or recorded on a computer readable medium for use in calculating for example editing rates for a genome editing experiment.

Genome editing with engineered nucleases, usually simply referred to as ‘genome editing’, is a process of inserting, deleting, or modifying genomic sequences using sequence-specific nucleases. Several methods for genome editing currently exist, including meganucleases, zinc-finger nucleases (ZFNs), transcription activator-like effector-based nucleases (TALENs), and the CRISPR-Cas system. These nucleases may induce double-stranded DNA breaks that can subsequently be repaired by two cellular pathways: either non-homologous end joining (NHEJ), which typically results in inactivation of the gene through insertions or deletions (indels), and homology dependent repair (HDR), which allows for insertions or modifications of a gene using a template with homology to the DNA surrounding the double-stranded break.

FIG. 2 illustrates an example sequence alignment 200 that benefits from the biological informed alignment of FIG. 1 in the case of a genome editing experiment. Here we expect the genome editor to create a simple deletion between position 6 and 7, however multiple alignments with similar scores can be obtained with different strategies. Specifically, a plurality of example alignments 202-204 between a first sequence, ATCAGGTCGT, and a second sequence, AACATCGT. For each example alignment, a first scoring, representing a scoring system with a match incentive of +5, a mismatch penalty of −2, a gap opening penalty of −5, and a gap extension penalty of −1, is shown as the “Constant” score. A second scoring, representing a system with a match incentive of +5, a mismatch penalty of −2, a position-dependent gap opening penalty, and a gap extension penalty of −1, is shown as the “Position Dependent” score.

Looking at the three alignments 202-204, it will be appreciated that the gaps are allocated differently in each alignment, but the constant gap scoring results in the same score in each instance. This leads to ambiguity as in the alignment of the two sequences. The Position Dependent score uses biological knowledge to select alignments that have gaps at positions with high likelihood given our biological understanding based on an applied editing process. In the illustrated example, as mentioned above, it is assumed that the second sequence was read after application of a genome editing tool that creates a double-stranded break (resulting in insertions or deletions) and is targeted such that the cleavage enzyme is active immediately after the ‘CAAC’ sequence, indicated by the vertical dotted lines in FIG. 2. Accordingly, gaps after the ‘CAAC’ sequence are incentivized with a reduced gap opening penalty of −4. Due to the reduced penalty associated with the gap location, the second alignment is scored slightly higher than the other alignments in the Position Dependent scoring, which reflects a higher likelihood that the alignment is correct given our biological understanding of genome editing tool position and function.

FIG. 3 illustrates one implementation of a method 300 for aligning two sequences in accordance with an aspect of the present invention. Throughout this description, positions in the sequence A are indexed as k, positions in the sequence B are indexed as l. For example, the first sequence, A, could be a read sequence and the sequence, B, could be a reference sequence. The illustrated method 100 recursively constructs three matrices. In a first matrix, M, each element mₖₗ, represents a score of the best alignment between the segment A[0:k] and B[0:l] that ends with a match between Aₖ and Bₗ. In a second matrix, l, each element iₖₗ, represents a score of the best alignment between the segment A[0:k] and B[0:l] that ends with a gap in sequence B being paired with Aₖ. In a third matrix, J, each element jₖₗ, represents a score of the best alignment between the segment A[0:k] and B[0:l] that ends with a gap in sequence A being paired with Bₗ.

At 302, mismatch scores, sₖₗ, are calculated for at least a subset of the available combination of bases in sequences A and B. It will be appreciated that, in some implementations, not all mismatch scores will be required. Further, it will be appreciated that the mismatch between various bases can be set to different values depending on the implementation, such that different mismatches between bases are penalized to different degrees. Accordingly, the calculation of mismatch scores at 302 can be performed as needed during the method 300 as opposed to calculating them at the initiation of the sequence alignment. At 304, an incentive vector is populated. The incentive vector reflects existing knowledge of biological processes used to generate a particular sequence, such as where mutations to a genomic sequence are expected to occur. In one implementation, the incentive vector represents one or more predicted cut sites for a nuclease used to generate the genome represented by the read sequence. It will be appreciated that the inventive vector can contain a value for each position, k, along sequence A. It will be appreciated that, in some implementations, the incentive vector can be combined with the constant gap penalty, such that that the incentive vector represents a location-dependent gap penalty.

At 306, a value is generated for the element mₖₗ from the mismatch score, sₖₗ and the elements m_{k-1, l-1}, i_{k-1,l-1}, and j_{k-1,l-1}. In one example, a maximum value is selected from the elements m_{k-1, l-1}, i_{k-1,l-1}, and j_{k-1,l-1} and added to the mismatch score, sₖₗ, to provide mₖₗ. At 308, a value is generated for the element iₖₗ from a gap opening penalty, GO, a gap extension penalty, GP, the incentive vector, GI, and the elements m_{k, l-1} and i_{k,l-1}. Each of the gap opening penalty and the gap extension penalty are, as the name would suggest, penalties on the fitness of a given alignment, but in practice, the gap extension penalty tends to be smaller than the gap opening penalty to reflect the biological intuition that insertions and deletions are caused for example by a single nuclease cleavage event rather than multiple cleavages that create multiple single-base deletions or insertions. However, the scores can be set based on the particular biological process in consideration. In one example iₖₗ is determined as the larger of the sum of the gap opening penalty, the value for the incentive vector at position k, and the element m_{k, l-1} and the sum of the gap extension penalty, the value for the incentive vector at position k, and the element j_{k,l-1}.

At 310, a value is generated for the element jₖₗ from the gap opening penalty, the gap extension penalty, the incentive vector, and the elements m_{k-1, l} and j_{k-l,1}. In one example jₖₗ is determined as the larger of the sum of the gap opening penalty, the value for the incentive vector at position k-1, and the element m_{k-1,l} and the sum of the gap extension penalty, the value for the incentive vector at position k-1, and the element i_{k-1,l}. At 312, it is determined if all matrices M, I, and J have been fully populated. If not (N), the method returns to 306, with one the indices k or l advanced by one. Once the matrices have been fully populated (Y), a traceback procedure is performed at 314 to determine an optimal alignment between the two sequences. In one example, the traceback first identifies a highest-valued entry, M(x, y) in the matrix M. The entries in H are traced sequentially from the highest-valued entry to a highest valued neighbor associated with a previous location in one sequence [M(x-1, y), M(x, y-1), or M(x-1, y-1)] until a zero entry is reached. The resulting trace will originate at the highest-valued entry and extend “back” to its terminus at the first zero entry encountered. In another example, the traceback begins at the bottom-right position in matrix M, and follows the path of the entries that produced that score backwards until the top-left position is reached. That trace indicates the optimally-scoring alignment between the sequences or two subsequences of the sequences. The determined optimal-scoring alignment can then be displayed to a user at an associated display or recorded on a computer readable medium for use in calculating an edit rate for the genome editing process.

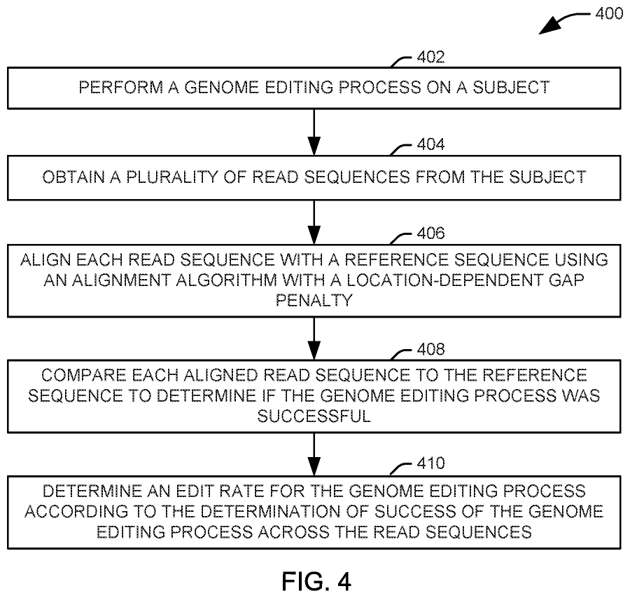

FIG. 4 illustrates a method 400 for evaluating the effectiveness of a genome editing process in accordance with an aspect of the present invention. The method 400 begins at 402, where a genome editing process is performed on a subject, where the subject can be DNA, RNA, or protein from a human, a cell line, or other source of DNA, RNA, or protein that can be measured and represented by a sequence of symbols on a given alphabet. Traditionally, genome editing has been performed by transfecting or transducing cells with RNA or DNA that then produce the proteins, and in the case of the CRISPR-Cas system, the guide RNAs required for genome editing. The technologies described herein are independent of the genome editing tool used, and may be applied to currently known genome editing modalities, as well as future novel methods that are used to produce changes in DNA, RNA, or protein in a programmable manner.

At 404, a plurality of read sequences are obtained from the subject. Sequence reads may be obtained by any suitable means. in certain embodiments, a nucleic add is isolated and sequenced. Nucleic acid template molecules, such as DNA or RNA, can be isolated from a sample containing other components, such as proteins, lipids and non-template nucleic acids. Nucleic add can be obtained from a tissue or body fluid specimen or cell sample taken from the subject, or from another source such as a cell line or synthesized nucleic adds. Generally, nucleic add can be extracted, isolated, amplified, or analyzed by a variety of techniques.

Nucleic acid obtained from biological samples may be fragmented to produce suitable fragments for analysis. Template nucleic acids may be fragmented or sheared to desired length, using a variety of mechanical, chemical and/or enzymatic methods. Nucleic add may be sheared by sonication, brief exposure to a DNase/RNase, HydroShear instrument, one or more restriction enzymes, transposase or nicking enzyme, exposure to heat plus magnesium, or by mechanical shearing. RNA may be converted to cDNA, for example, before or after fragmentation. A sample may be lysed, homogenized, or fractionated in the presence of a detergent or surfactant by methods known in the art.

Nucleic add may also be amplified. Amplification refers to production of additional copies of a nucleic add sequence and is generally carried out using polymerase chain reaction (PCR) or other technologies known in the art. The amplification reaction may be any amplification reaction known in the art that amplifies nucleic add molecules, such as PCR, nested PCR, PCR-single strand conformation polymorphism, and rolling circle amplification. Further examples of amplification include reverse transcriptase PCR, quantitative PCR, quantitative fluorescent PCR (QF-PCR), multiplex fluorescent PCR (MF-PCR), real time PCR (RTPCR), restriction fragment length polymorphism PCR (PCR-RFLP), bridge PCR, picotiter PCR, emulsion PCR, consensus sequence primed PCR, arbitrarily primed PCR, and degenerate oligonucleotide-primed PCR.

With these methods, target nucleic add may be amplified and sequenced. Sequencing may be done by any suitable method including, for example, Sanger sequencing using labeled terminators or primers and gel separation in slab or capillary, sequencing by synthesis using reversibly terminated labeled nucleotides, pyrosequencing, the single molecule, real-time sequencing, 454 sequencing, Illumina sequencing, ion semiconductor sequencing, nanopore sequencing, real time monitoring of the incorporation of labeled nucleotides during a polymerization step, polony sequencing, SOLID sequencing, sequencing methods using a chemically-sensitive field-effect transistor, and sequencing methods using an electron microscope. Separated molecules may be sequenced by sequential or single extension reactions using polymerases or ligases as well as by single or sequential differential hybridizations with libraries of probes.

Once the read sequences are obtained, each read sequence is aligned with a reference sequence, representing the subject's genome prior to the genome editing at 402, using an alignment algorithm employing a location-dependent gap penalty at 406. For example, the alignment of each read sequence with the reference sequence via the method illustrated in FIG. 1 or the method illustrated in FIG. 3. At 408, each aligned read sequence is compared to the reference sequence to determine if the genome editing process at 402 was successful. At 410, an edit rate for the genome editing process is determined according to the determination of the success of the genome editing process across the plurality of read sequences. The edit rate can then be displayed to a user at an associated display and/or stored on a computer readable medium, for example, as part of a patient record. Other information such as the rates and locations of substitutions, insertions, or deletions, may be recorded and displayed to the user at an associated display and/or stored on a computer readable medium, for example, as part of a patient record.

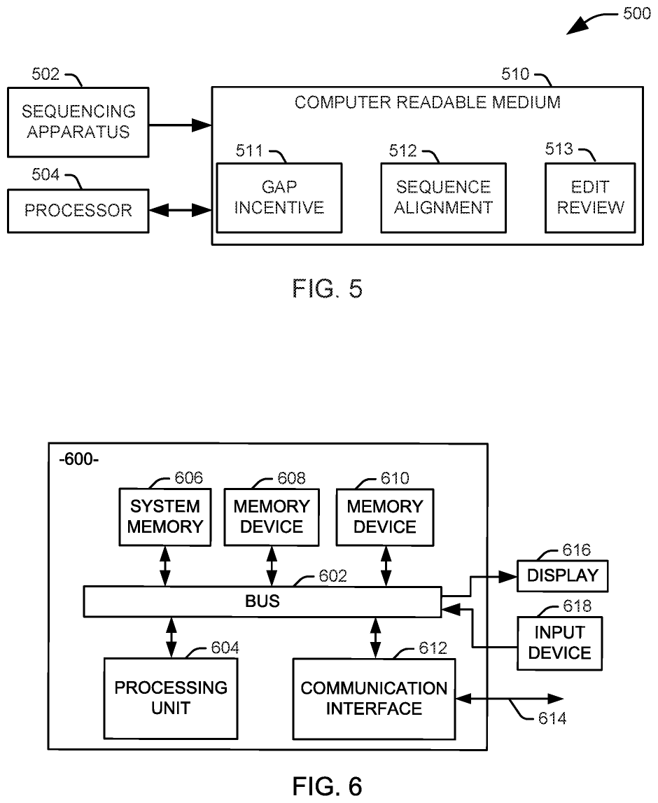

FIG. 5 illustrates a system 500 in accordance with an aspect of the present invention. The system 500 includes a sequencing apparatus 502, a processor 504, and a non-transitory computer readable medium 510 storing executable instructions 511-513. The sequencing apparatus 502 can include any appropriate hardware for obtaining nucleic acid sequences from a sample taken from a subject. It will be appreciated that the specific hardware for the sequencing apparatus 502 will vary with the sequencing method applied.

The executable instructions include a gap incentive component 511 that populates a gap vector having a plurality of gap penalty values representing a respective plurality of locations along a first sequence according to expected locations for an action of a biological process applied to the subject. The various elements of the gap vector can be selected to represent biological information about the likelihood of a mutation occurring at each location in the first sequence. In one implementation, in which the biological process is a genome editing process that creates a double-stranded break and is targeted such that an associated cleavage enzyme is active at a certain position in the sequence, the gap incentive component 511 populates the gap vector with a reduced gap penalty value for any location immediately following the specific sequence. Alternatively, mismatch matrices can be also derived based on positional preferences of other agents such as base editors that preferentially create base substitutions in specific location of a sequence.

A sequence alignment component 512 generates, for each of a set of possible alignments of the first sequence and the second sequence, a score representing the fitness of each possible alignment according to at least a match incentive, a mismatch penalty, and the gap vector and selects a possible alignment of the set of possible alignments having a best score as an alignment between the first sequence and the second sequence. In one implementation, the sequence alignment component generates the score by iteratively populating a matrix such that each element of the at least one matrix representing the fitness of a possible alignment, and selects the possible alignment having the best score by performing a traceback procedure of the matrix to determine an optimal alignment of the first sequence to the second sequence.

It will be appreciated that the system 500 can be used to compare arbitrary sequences. In one example, however, the system can be employed to evaluate the results of a genome editing process on a subject. In such a case, a reference sequence can be acquired from the subject prior to application of the genome editing process as the first sequence. One or more cell samples can be extracted from the patient and sequenced to provide a set of read sequences. Each read sample can be aligned with the reference sample at the sequence alignment component 512. An edit review component 513 then determines, from the alignment of each of the set of read sequences with the first sequence, if the read sequence represents a successful application of the genome editing process. Once this is done for each read sequence, an edit rate across the set of read sequences can be determined from a number of read sequences of the set of read sequences that represent successful applications of the genome editing process. For example, the edit rate can be computed as a ratio of the number of read sequences representing successes to a total number of read sequences. It is worth mentioning that this process involves thousands to million of sequences and it cannot be performed manually in a reasonable time.

FIG. 6 is a schematic block diagram illustrating an exemplary system 600 of hardware components capable of implementing examples of the systems and methods disclosed in FIGS. 1-5. The system 600 can include various systems and subsystems. The system 600 can be a personal computer, a laptop computer, a workstation, a computer system, an appliance, an application-specific integrated circuit (ASIC), a server, a server blade center, a server farm, etc.

The system 600 can includes a system bus 602, a processing unit 604, a system memory 606, memory devices 608 and 610, a communication interface 612 (e.g., a network interface), a communication link 614, a display 616 (e.g., a video screen), and an input device 618 (e.g., a keyboard and/or a mouse). The system bus 602 can be in communication with the processing unit 604 and the system memory 606. The additional memory devices 608 and 610, such as a hard disk drive, server, stand-alone database, or other non-volatile memory, can also be in communication with the system bus 602. The system bus 602 interconnects the processing unit 604, the memory devices 606-610, the communication interface 612, the display 616, and the input device 618. In some examples, the system bus 602 also interconnects an additional port (not shown), such as a universal serial bus (USB) port.

The processing unit 604 can be a computing device and can include an application-specific integrated circuit (ASIC). The processing unit 604 executes a set of instructions to implement the operations of examples disclosed herein. The processing unit can include a processing core.

The additional memory devices 606, 608 and 610 can store data, programs, instructions, database queries in text or compiled form, and any other information that can be needed to operate a computer. The memories 606, 608 and 610 can be implemented as computer-readable media (integrated or removable) such as a memory card, disk drive, compact disk (CD), or server accessible over a network. In certain examples, the memories 606, 608 and 610 can comprise text, images, video, and/or audio, portions of which can be available in formats comprehensible to human beings. Additionally or alternatively, the system 600 can access an external data source or query source through the communication interface 612, which can communicate with the system bus 602 and the communication link 614.

In operation, the system 600 can be used to implement one or more parts of a sequence alignment system or method in accordance with the present invention. Computer executable logic for implementing the sequence alignment resides on one or more of the system memory 606, and the memory devices 608, 610 in accordance with certain examples. The processing unit 604 executes one or more computer executable instructions originating from the system memory 606 and the memory devices 608 and 610. The term “computer readable medium” as used herein refers to any medium that participates in providing instructions to the processing unit 604 for execution, and it will be appreciated that a computer readable medium can include multiple computer readable media each operatively connected to the processing unit.

Specific details are given in the above description to provide a thorough understanding of the embodiments. However, it is understood that the embodiments can be practiced without these specific details. For example, physical components can be shown in block diagrams in order not to obscure the embodiments in unnecessary detail. In other instances, well-known circuits, processes, algorithms, structures, and techniques can be shown without unnecessary detail in order to avoid obscuring the embodiments.

Implementation of the techniques, blocks, steps and means described above can be done in various ways. For example, these techniques, blocks, steps and means can be implemented in hardware, software, or a combination thereof. For a hardware implementation, the processing units can be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above, and/or a combination thereof.

Also, it is noted that the embodiments can be described as a process which is depicted as a flowchart, a flow diagram, a data flow diagram, a structure diagram, or a block diagram. Although a flowchart can describe the operations as a sequential process, many of the operations can be performed in parallel or concurrently. In addition, the order of the operations can be re-arranged. A process is terminated when its operations are completed, but could have additional steps not included in the figure. A process can correspond to a method, a function, a procedure, a subroutine, a subprogram, etc. When a process corresponds to a function, its termination corresponds to a return of the function to the calling function or the main function.

Furthermore, embodiments can be implemented by hardware, software, scripting languages, firmware, middleware, microcode, hardware description languages, and/or any combination thereof. When implemented in software, firmware, middleware, scripting language, and/or microcode, the program code or code segments to perform the necessary tasks can be stored in a machine readable medium such as a storage medium. A code segment or machine-executable instruction can represent a procedure, a function, a subprogram, a program, a routine, a subroutine, a module, a software package, a script, a class, or any combination of instructions, data structures, and/or program statements. A code segment can be coupled to another code segment or a hardware circuit by passing and/or receiving information, data, arguments, parameters, and/or memory contents. Information, arguments, parameters, data, etc. can be passed, forwarded, or transmitted via any suitable means including memory sharing, message passing, ticket passing, network transmission, etc.

For a firmware and/or software implementation, the methodologies can be implemented with modules (e.g., procedures, functions, and so on) that perform the functions described herein. Any machine-readable medium tangibly embodying instructions can be used in implementing the methodologies described herein. For example, software codes can be stored in a memory. Memory can be implemented within the processor or external to the processor. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium” can represent one or more memories for storing data, including read only memory (ROM), random access memory (RAM), magnetic RAM, core memory, magnetic disk storage mediums, optical storage mediums, flash memory devices and/or other machine readable mediums for storing information. The term “machine-readable medium” includes, but is not limited to portable or fixed storage devices, optical storage devices, wireless channels, and/or various other storage mediums capable of storing that contain or carry instruction(s) and/or data.

What have been described above are examples of the present invention. It is, of course, not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, modifications, and variations that fall within the scope of the appended claims.

What is claimed is:

1. A computer-implemented method for aligning a first sequence and a second sequence comprising:
populating a gap vector having a plurality of gap penalty values representing a respective plurality of locations along the first sequence, such that a first gap penalty value associated with a first location of the plurality of locations is different than a second gap penalty value associated with a second location of the plurality of locations;
generating, for each of a set of possible alignments of the first sequence and the second sequence, a score representing the fitness of each possible alignment, the score for each possible alignment being determined according to at least a match incentive, a mismatch penalty, and the gap vector; and
selecting a possible alignment of the set of possible alignments having a best score as an alignment between the first sequence and the second sequence.

2. The computer-implemented method of claim 1, wherein generating the score representing the fitness of each possible alignment comprises iteratively populating a matrix such that each element of the at least one matrix is the score representing the fitness of a possible alignment, and selecting the possible alignment having the best score as the alignment between the first sequence and the second sequence comprises performing a traceback procedure of the matrix to determine an optimal alignment of the first sequence to the second sequence.

3. The computer-implemented method of claim 1, wherein populating the gap vector comprises selecting the plurality of gap penalty values representing the respective plurality of locations along the first sequence according to expected locations for an action of a biological process applied to a subject associated with the first sequence and the second sequence.

4. The computer-implemented method of claim 1, wherein the biological process is a genome editing process.

5. The computer-implemented method of claim 4, wherein the genome editing process creates a modification and is targeted such that an associated enzyme is active at a certain location in the sequence, and populating the gap vector comprises selecting a reduced gap penalty value for the location in the sequence corresponding to the predicted editing site of the cleavage enzyme.

6. The computer implemented method of claim 1, wherein the gap vector represents a location-dependent gap opening penalty, and the score for each possible alignment is determined according to the match incentive, the mismatch penalty, and the gap vector.

7. The computer implemented method of claim 1, wherein the gap vector represents a location-dependent incentive applied according to the location of a gap on the first sequence, and the score for each possible alignment is determined according to the match incentive, the mismatch penalty, a constant gap opening penalty, and the gap vector.

8. The computer implemented method of claim 1, further comprising performing the genome editing process on the subject, wherein populating the gap vector comprises selecting the plurality of gap penalty values representing the respective plurality of locations along the first sequence according to expected locations for an action of the genome editing process.

9. The computer implemented method of claim 8, wherein the first sequences a reference sequence for the subject and the method further comprises acquiring a plurality of read sequences from the subject, the second sequence being one of the plurality of read sequences.

10. The computer-implemented method of claim 9, further comprising:
determining, from the alignment of each of the plurality of read sequences with the first sequence, if the read sequence represents a successful application of the genome editing process, and recording the locations and types of genome editing events in the read sequences; and
determining an edit rate across the plurality of read sequences indicative of the number of read sequences of the plurality of read sequences that represent successful applications of the genome editing process.

11. A system comprising:
a processor; and
a non-transitory computer readable medium storing instructions, executable by the processor, for aligning a first sequence and a second sequence, said executable instructions comprising:
a gap incentive component that populates a gap vector having a plurality of gap penalty values representing a respective plurality of locations along the first sequence according to expected locations for an action of a biological process applied to a subject associated with the first sequence and the second sequence; and
a sequence alignment component that generates, for each of a set of possible alignments of the first sequence and the second sequence, a score representing the fitness of each possible alignment according to at least a match incentive, a mismatch penalty, and the gap vector and selects a possible alignment of the set of possible alignments having a best score as an alignment between the first sequence and the second sequence.

12. The system of claim 11, wherein the sequence alignment component generates the score representing the fitness of each possible alignment by iteratively populating a matrix such that each element of the at least one matrix is the score representing the fitness of a possible alignment, and selects the possible alignment having the best score by performing a traceback procedure of the matrix to determine an optimal alignment of the first sequence to the second sequence.

13. The system of claim 11, wherein the biological process is a genome editing process.

14. The system of claim 13, wherein the genome editing process creates a double-stranded break and is targeted such that an associated cleavage enzyme is active immediately after a specific sequence of nucleotides, and the gap incentive component populates the gap vector comprises selecting a reduced gap penalty value for any location immediately following the specific sequence.

15. The system of claim 14, further comprising a sequencing apparatus to acquire a set of read sequences, the set of read sequences comprising the second sequence.

16. The system of claim 14, wherein the executable instructions further comprise a edit review component determining, from the alignment of each of the set of read sequences with the first sequence, if the read sequence represents a successful application of the genome editing process and determines an edit rate across the set of read sequences indicative of the number of read sequences of the set of read sequences that represent successful applications of the genome editing process, and the locations and types of genome editing modifications in the read sequences.

17. A method for determining an edit rate for a genome editing process comprising:
performing the genome editing process on the subject;
acquiring a plurality of read sequences from the subject;
populating a gap vector having a plurality of gap penalty values representing a respective plurality of locations along a reference sequence associated with the subject, such that a first gap penalty value associated with a first location of the plurality of locations is different than a second gap penalty value associated with a second location of the plurality of locations;
aligning each of the plurality of read sequences with a reference sequence, wherein aligning each read sequence with the reference sequence comprises:
generating, for each of a set of possible alignments of the reference sequence and the read sequence, a score representing the fitness of each possible alignment, the score for each possible alignment being determined according to at least a match incentive, a mismatch penalty, and the gap vector; and
selecting a possible alignment of the set of possible alignments having a best score as an alignment between the reference sequence and the read sequence;
determining, from the alignment of each read sequence with the reference sequence, if the read sequence represents a successful application of the genome editing process;
determining an edit rate across the plurality of read sequences indicative of the number of read sequences of the plurality of read sequences that represent successful applications of the genome editing process; and
determining the locations and types of genome editing modifications in the read sequences.

18. The method of claim 17, wherein generating the score representing the fitness of each possible alignment comprises iteratively populating a matrix such that each element of the at least one matrix is the score representing the fitness of a possible alignment, and selecting the possible alignment having the best score as the alignment between the reference sequence and the read sequence comprises performing a traceback procedure of the matrix to determine an optimal alignment of the first sequence to the second sequence.

19. The method of claim 17, wherein populating the gap vector comprises selecting the plurality of gap penalty values representing the respective plurality of locations along the read sequence according to expected locations for an action of the genome editing process.

20. The method of claim 19, wherein genome editing process creates a double-stranded break and is targeted such that an associated cleavage enzyme is active at a specific location in the sequence, and populating the gap vector comprises selecting a reduced gap penalty value for any location targeted by the genome editing process.
